(19) United States
(12) Patent Application Publication
(10) Pub. No.: US 2013/0184316 A1
(43) Pub. Date: Jul. 18, 2013

(54) METHODS FOR DIAGNOSING AND TREATING CONCUSSIVE DISORDERS

(71) Applicant: Andrew Hornstein, New City, NY (US)

(72) Inventor: Andrew Hornstein, New City, NY (US)

(21) Appl. No.: 13/810,280

(22) PCT Filed: Jul. 7, 2011
(86) PCT No.: PCT/US11/43186

§ 371 Date: Apr. 4, 2013

Related U.S. Application Data
(60) Provisional application No. 61/364,665, filed on Jul. 15, 2010.

(51) Int. Cl. A61B 5/00; A61B 5/11; A61K 31/4409

(52) U.S. Cl. CPC A61B 5/4848; A61K 31/4409; A61B 5/11
USPC 514/352; 600/301

(57) ABSTRACT

The invention provides methods for treating, suppressing, ameliorating and/or preventing the effects of disorders relating to concussion, including those associated with post-concussion syndrome. The methods comprise administering to a subject an effective amount of a compound that modulates neuronal ion channel function, or a pharmaceutically acceptable salt, solvate or hydrate thereof, or a composition comprising a compound that modulates neuronal ion channel function, or a pharmaceutically acceptable salt, solvate or hydrate thereof, and a pharmaceutically acceptable carrier. The invention also provides inventive methods relating to identifying, classifying and/or diagnosing conditions involving post-concussion syndrome and related concussive disorders.

This application claims benefit of priority to U.S. Provisional Patent Application No. 61/364,665, filed Jul. 15, 2010, the entire disclosure of which is hereby incorporated by reference herein.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Concussion is a form of traumatic head injury that refers to an immediate and transient loss or perturbation of consciousness accompanied by a brief period of amnesia after a blow to the head. Concussion is a major public health concern, with over 1 million new cases annually in the United States. It is also the most common injury of American combat personnel. Despite its prevalence, the pathophysiology underlying concussion remains largely unknown. Some have postulated various mechanisms to explain the host of symptoms associated with concussion, including temporary disruption of the reticular activating system, self-limiting seizure activity, transient changes in intracranial pressure, release of inhibitory neurotransmitters, and perturbations of local ion concentrations and metabolic rates, all resulting from sudden rotational forces on the cerebral hemispheres.

Although most patients with concussion experience resolution of symptoms within days to weeks, it has been recognized by those of skill in the art that a significant minority of patients have reported persistent, disabling symptoms after a concussive event. Even moderate concussive head injury can result in persistent neurological disturbances that often last for months. These disturbances frequently include dizziness, balance problems, and fine motor skill dysfunction (Lyeth et al., Brain Res. 1988, 452, 39-48; hereby incorporated by reference in its entirety). The art has defined several labels to capture patients diagnosed as exhibiting such persistent symptoms, including postconcussion syndrome (“PCS”), post-concussive disorder (“PCD”) and residual mild traumatic brain injury.

PCS is characterized by a wide variety of symptoms, principally including headache, dizziness, and trouble concentrating in the days and weeks following concussion. Both the International Statistical Classification of Diseases and Related Health Problems, 10th Rev. (“ICD-10”) and Diagnostic and Statistical Manual of Mental Disorders, 4th Ed. (“DSM-IV”), both incorporated herein by reference in their entireties, include provisional diagnostic criteria for PCS. Both authorities identify a patient with PCS as having a history of head trauma that has caused a significant cerebral concussion frequently resulting in loss of consciousness or post-traumatic amnesia. Referring specifically to the DSM-IV, the patient exhibits difficulty in attention (i.e., difficulty concentrating, shifting focus of attention, performing simultaneous cognitive tasks) or memory, as measured objectively by neurophysiological testing or quantified cognitive assessment. In addition, the patient will often exhibit three or more of the following symptoms that last at least 3 months after the trauma: becoming fatigued easily, disordered sleep, headache, vertigo or dizziness, irritability or aggression on little or no provocation, anxiety, depression or affective liability, changes in personality, and apathy or lack of spontaneity. These symptoms typically cause a significant impairment in social and/or occupational functioning.

The vague and often subjective nature of the diagnostic criteria pose problems with proper PCS diagnosis, however. In addition, PCS symptoms overlap with symptoms of with those of post-traumatic stress disorder and depression, and may also be masked by substance abuse. Another difficulty in the diagnosis and understanding of PCS is the fact that, thus far, the putative neurological injury is below the threshold of detection of clinically available imaging technology, and not consistently or usefully within the scope of newer techniques such as diffusion tensor imaging and magnetoencephlography.

Nevertheless, epidemiological studies have suggested the subset of concussive patients who go on to develop PCS may be as large as 44% (See, e.g., McCrea M. A. (2008) Mild Traumatic Brain Injury and Postconcussion Syndrome. New York: Oxford University Press; Lannsjo M. et al., Brain Inj., 2009, 23:213-219; each of which is hereby incorporated by reference in its entirety). Like the etiology of concussion itself, it is currently unknown why a subset of patients will go on to develop PCS.

There is therefore a need in the art to reliably identify patients having PCS and, once identified, provide useful treatments and therapies to alleviate symptoms in patients in need.

### SUMMARY OF THE INVENTION

The invention is directed to methods and compositions for treating, suppressing and/or preventing concussive disorders such as, for example, post concussion syndrome, or afflictions related thereto, in subjects; and methods for identification, classification and/or diagnosis of subjects that are candidates for treatment, suppression, and/or prevention of concussive disorders such as, for example, post concussion syndrome, or afflictions related thereto.

In one embodiment, the inventive methods identify, classify and/or diagnose subjects who, based on qualitative and quantitative features of their symptoms, are likely to benefit from treatment with neuronal ion channel blockers to ameliorate PCS symptoms. In one embodiment, identification, classification and/or diagnosis is performed by evaluation of a physician. In one embodiment, evaluation is neurological. In one embodiment, evaluation is neuro-optometric. In one embodiment, evaluation is neuro-otological. In one embodiment, evaluation is neuro-physiological. In one embodiment, evaluation is neuro-psychological. In one embodiment, evaluation is neuro-ophthamologicial. In one embodiment, evaluation is psychological. In one embodiment, evaluation is physical. In one embodiment, evaluation is psychiatric.

In one embodiment, the methods comprise administering to the subject in need thereof a therapeutically effective amount of a compound, or a pharmaceutically acceptable salt, solvate or hydrate thereof, that modulates or blocks neuronal ion channel function, or a composition comprising a compound, or a pharmaceutically acceptable salt, solvate or hydrate thereof, that modulates or blocks neuronal ion channel function and a pharmaceutically acceptable carrier.

In one embodiment, the methods treat post concussion syndrome or symptoms related thereto. In one embodiment, the methods reduce post concussion syndrome or symptoms related thereto. In one embodiment, the methods suppress post concussion syndrome or symptoms related thereto. In one embodiment, the methods identify post concussion syndrome. In one embodiment, the methods classify post concussion syndrome. In one embodiment, the methods diagnose post concussion syndrome.

In one embodiment, the compound modulates voltage-gated potassium channels. In one embodiment, the compound blocks voltage-gated potassium channels.

In one embodiment, the compound is a mono- or di-aminopyridine, or a pharmaceutically acceptable salt, solvate or hydrate thereof. In one embodiment, the compound is 4-aminopyridine. In one embodiment, the compound is 3,4-diaminopyridine.

In one embodiment, the subject exhibits symptoms selected from at least two symptom clusters selected from the group consisting of balance impairments, vertigo, cognitive or emotional impairments, and somatosensory impairments. In another embodiment, the subject exhibits symptoms selected from at least three symptom clusters selected from the group consisting of balance impairments, vertigo, cognitive or emotional impairments, and somatosensory impairments. In yet another embodiment, the subject exhibits at least one symptom from each of the following symptom clusters: balance impairments, vertigo, cognitive or emotional impairments, and somatosensory impairments. In still another embodiment, the subject exhibits symptoms of post-traumatic stress disorder, vertigo, depression, or substance abuse.

In one embodiment, the subject is suffering from treatment-resistant post concussion syndrome. In another embodiment, the subject has partially responded to treatment for traumatic brain injury. In another embodiment, the subject has partially responded to treatment for concussive disorder.

In one embodiment, the present invention provides a method for identifying, classifying and/or diagnosing subjects afflicted with post concussion syndrome. In one embodiment, the present invention provides a method for diagnosing post concussion syndrome. In one embodiment, the methods are useful for identifying subjects suffering from PCS that may be candidates for treatment with a compound that modulates neuronal ion channel function. In one embodiment, the methods are useful for classifying subjects suffering from PCS that may be candidates for treatment with a compound that modulates neuronal ion channel function. In one embodiment, the methods are useful for diagnosing subjects suffering from PCS that may be candidates for treatment with a compound that modulates neuronal ion channel function.

In one embodiment, the methods for identifying, classifying and/or diagnosing subjects in need thereof comprise determining that the subject exhibits symptoms selected from at least two system clusters selected from the group consisting of symptoms related to balance impairments, symptoms related to vertigo, symptoms related to cognitive or emotional impairments, and symptoms related to somatosensory impairments. In one embodiment, the methods further comprise administering to the subject a therapeutically effective amount of a compound, or a pharmaceutically acceptable salt, solvate or hydrate thereof, that modulates or blocks neuronal ion channel function, or a composition comprising a compound, or a pharmaceutically acceptable salt, solvate or hydrate thereof, that modulates or blocks neuronal ion channel function and a pharmaceutically acceptable carrier. In another embodiment, the subject has experienced head or brain injury prior to determination of symptoms.

In one embodiment, the methods for treating, suppressing and/or preventing post concussion syndrome in subjects in need thereof comprise a) determining that the subject exhibits symptoms selected from at least two system clusters selected from the group consisting of symptoms related to balance impairments, symptoms related to vertigo, symptoms related to cognitive or emotional impairments, and symptoms related to somatosensory impairments; and b) administering to the subject a therapeutically effective amount of a compound or a pharmaceutically acceptable salt, solvate, or hydrate thereof, or a composition comprising a compound or a pharmaceutically acceptable salt, solvate, or hydrate thereof and a pharmaceutically acceptable carrier, wherein the compound modulates or blocks that modulates neuronal ion channel function. In one embodiment, the determining step is performed by evaluation of a physician. In one embodiment, the evaluation is neurological, neuro-optometric, neuro-otological, neuro-physiological, neuro-psychological, psychological, neuro-ophthamologicial, psychiatric or physical. In one embodiment, evaluation is neurological. In one embodiment, evaluation is neuro-optometric. In one embodiment, evaluation is neuro-otological. In one embodiment, evaluation is neuro-physiological. In one embodiment, evaluation is neuro-psychological. In one embodiment, evaluation is neuro-ophthamologicial. In one embodiment, evaluation is psychological. In one embodiment, evaluation is physical. In one embodiment, evaluation is psychiatric.

In one embodiment, the subject is a mammal. In another embodiment, the subject is a mouse, rat, monkey, guinea pig, dog, or human. In yet another embodiment, the subject is a mouse, rat, monkey or human. In still another embodiment, the subject is a mouse or a human. In still another embodiment, the subject is a human.

In one embodiment, the methods treat, suppress and/or prevent symptoms related to post concussion syndrome. In another embodiment, the methods treat, suppress and/or prevent vertigo, balance impairments, cognitive or emotional impairments, and/or somatosensory impairments. In still another embodiment, the methods treat, suppress and/or prevent vertigo, disequilibrium, difficulty with static or mobile vision, and/or difficulty with integration of multiple stimuli.

The present invention is based, in part, on certain discoveries which are described more fully in the Examples section of the present application. For example, the present invention is based, in part, on the discovery that symptoms related to post concussion syndrome were markedly reduced upon treatment with a compound that modulates neuronal ion channel function.

These and other embodiments of the invention are further described in the following sections of the application, including the Detailed Description, Examples, and Claims. Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

### DETAILED DESCRIPTION OF THE INVENTION

The invention is directed to methods and compositions for treating, suppressing and/or preventing post concussion syndrome, or afflictions related thereto, in subjects; and methods for identification, classification and/or diagnosis of subjects that are candidates for treatment, suppression, and/or prevention of post concussion syndrome, or afflictions related thereto.

In one embodiment, the inventive methods identify, classify and/or diagnose subjects who, based on qualitative and quantitative features of their symptoms, are likely to benefit from treatment with neuronal ion channel blockers to ameliorate PCS symptoms. The prevalence and social and economic costs of post-concussive symptoms, particularly in populations such as, for example, military personnel, exemplify a need for treatment of such. The methods of the present invention provide useful methods of treatment in a population in need thereof. Other compounds having similar therapeutic profiles are expected to be similarly useful in the treatment of subjects suffering from PCS.

In one embodiment, the methods comprise administering to the subject in need thereof a therapeutically effective amount of a compound, or a pharmaceutically acceptable salt, solvate or hydrate thereof, that modulates or blocks neuronal ion channel function, or a composition comprising a compound, or a pharmaceutically acceptable salt, solvate or hydrate thereof, that modulates or blocks neuronal ion channel function and a pharmaceutically acceptable carrier.

In one embodiment, the compound modulates voltage-gated potassium channels. In one embodiment, the compound blocks voltage-gated potassium channels. In one embodiment, the compound is a potassium channel blocker. In certain embodiments a potassium channel blocker includes, for example, acetazolamide, amantadine, apamin, charybdotoxin, chromanol 293B, CP 339818 hydrochloride, dequalinium dichloride, di-aminopyridines (such as 3,4-diaminopyridine), DMP 543, DPO1, edrophonium chloride, E-4031 dihydrochloride, iberiotoxin, KN-93, linopirdine dihydrochloride, mono-aminopyridines (such as 4-aminopyridine), paxilline, phencyclidine, procaine, quinidine, tetraethylammonium bromide, tetraethylammonium chloride, XE 991 dihydrochloride, or YS-035 hydrochloride.

In one embodiment, the compound is a mono- or di-aminopyridine, or a pharmaceutically acceptable salt, solvate or hydrate thereof. In one embodiment, the compound is 4-aminopyridine, or a pharmaceutically acceptable salt, solvate or hydrate thereof. In one embodiment, the compound is 3,4-diaminopyridine, or a pharmaceutically acceptable salt, solvate or hydrate thereof.

In one embodiment, the present invention provides a method for identifying, classifying and/or diagnosing subjects afflicted with post concussion syndrome. In one embodiment, the present invention provides a method for diagnosing post concussion syndrome. In one embodiment, the methods are useful for identifying subjects suffering from PCS that may be candidates for treatment with a compound that modulates or blocks neuronal ion channel function.

In one embodiment, the methods for identifying, classifying and/or diagnosing subjects in need thereof comprise determining that the subject exhibits symptoms selected from at least two system clusters selected from the group consisting of symptoms related to balance impairments, symptoms related to vertigo, symptoms related to cognitive or emotional impairments, and symptoms related to somatosensory impairments. In one embodiment, the methods further comprise administering to the subject a therapeutically effective amount of a compound, or a pharmaceutically acceptable salt, solvate or hydrate thereof, that modulates or blocks neuronal ion channel function, or a composition comprising a compound, or a pharmaceutically acceptable salt, solvate or hydrate thereof, that modulates or blocks neuronal ion channel function and a pharmaceutically acceptable carrier

In one embodiment, the methods for treating, suppressing and/or preventing post concussion syndrome in subjects in need thereof comprise a) determining that the subject exhibits symptoms selected from at least two system clusters selected from the group consisting of symptoms related to balance impairments, symptoms related to vertigo, symptoms related to cognitive or emotional impairments, and symptoms related to somatosensory impairments; and b) administering to the subject a therapeutically effective amount of a compound or a pharmaceutically acceptable salt, solvate, or hydrate thereof, or a composition comprising a compound or a pharmaceutically acceptable salt, solvate, or hydrate thereof and a pharmaceutically acceptable carrier, wherein the compound modulates or blocks that modulates neuronal ion channel function.

In one embodiment, the subject is a mammal. In another embodiment, the subject is a mouse, rat, monkey, guinea pig, dog, or human. In yet another embodiment, the subject is a mouse, rat, monkey or human. In still another embodiment, the subject is a mouse or a human. In still another embodiment, the subject is a human.

In one embodiment, the methods treat, suppress and/or prevent symptoms related to post concussion syndrome. In another embodiment, the methods treat, suppress and/or prevent vertigo, balance impairments, cognitive or emotional impairments, and/or somatosensory impairments. In still another embodiment, the methods treat, suppress and/or prevent vertigo, disequilibrium, difficulty with static or mobile vision, and/or difficulty with integration of multiple stimuli.

### ABBREVIATIONS AND DEFINITIONS

The term “post concussive syndrome” or “PCS” as used herein means a condition wherein a subject exhibits disabling, persistent symptoms that may be experienced after a concussion. The symptoms may include central nervous system imbalances and/or disorders such as, for example, dizziness, fatigue, personality changes, headaches, irritability, anxiety, noise sensitivity, impaired concentration, social withdrawal, decreased libido, and others. These and other symptoms related to PCS may be found, for example, listed in the Rivermead Post-Concussion Symptoms Questionaire (RPSCQ), which groups symptoms into three groups (mood and cognition symptoms, general somatic symptoms, and visual somatic symptoms) (J. Neuropsychiatry Clin. Neurosci. 2009, 21, 181-188; hereby incorporated by reference in its entirety). Both the International Statistical Classification of Diseases and Related Health Problems, 10th Rev. (“ICD-10”) and Diagnostic and Statistical Manual of Mental Disorders, 4th Ed. (“DSM-IV”), each of which hereby incorporated by reference in its entirety, include provisional diagnostic criteria for PCS. Both authorities identify a patient with PCS as having a history of head trauma that has caused a significant cerebral concussion frequently including loss of consciousness or post-traumatic amnesia. Referring specifically to the DSM-IV, the patient exhibits difficulty in attention (i.e., difficulty concentrating, shifting focus of attention, performing simultaneous cognitive tasks) or memory, measured objectively by neurophysiological testing or quantified cognitive assessment. In addition, the patient will often exhibit three or more of the following symptoms that last at least 3 months after the trauma: becoming fatigued easily; disordered sleep; headache; vertigo or dizziness; irritability or aggression on little or no provocation; anxiety, depression or affective liability; changes in personality; and apathy or lack of spontaneity. These symptoms typically cause a significant impairment in social or occupational functioning. Other descriptions of PCS symptoms may be found, for example, in references cited herein; and may be further evident to the skilled artisan. Accordingly, a subject with PCS may not exhibit each of the symptoms listed herein, but more typically a subset thereof. Often, the symptoms last for several months or, in some cases, years. Currently there is no effective treatment approved for PCS.

The term “compound of the invention” as used herein means a compound that blocks or modulates neuronal ion channel function. The terms are also intended to encompass salts, hydrates and solvates thereof. Representative compounds that modulate or block neuronal ion channel function include, for example, potassium channel blockers and calcium channel blockers.

The term “potassium channel blocker” as used herein is any agent that inserts itself into (or otherwise deactivates) any of the several and growing classes of potassium channels, including both voltage gated and non-gated channels. Representative potassium channel blockers include substituted pyridines and, more particularly, amino-substituted pyridines. In preferred embodiments, the methods as described herein involve the potassium channel blocker 4-AP (4-aminopyridine) or 3,4 di-aminopyridine.

The term “composition(s) of the invention” as used herein means compositions comprising a compound of the invention, and salts, hydrates, and solvates thereof. The compositions of the invention may further comprise other agents such as, for example, excipients, stabilants, lubricants, solvents, and the like.

The term “method(s) of the invention” as used herein means methods comprising treatment with the compounds and/or compositions of the invention, as well as methods comprising identifying, classifying and/or diagnosing PCS or subjects who are likely to benefit from or candidates for treatment for PCS.

The term “solvate” as used herein means a compound, or a pharmaceutically acceptable salt thereof, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

A “pharmaceutical composition” refers to a mixture of one or more of the compounds described herein, or pharmaceutically acceptable salts, solvates, or hydrates thereof, with other chemical components, such as physiologically acceptable carriers and excipients. The purpose of a pharmaceutical composition is to facilitate administration of a compound to an organism or subject.

The term “pharmaceutically acceptable salt” is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids; and salts derived from inorganic or organic bases including, for example sodium, potassium, calcium, ammonium or tetrafluoroborate. Exemplary pharmaceutically acceptable salts are found, for example, in Berge, et al. (J. Pharm. Sci. 1977, 66(1), 1; hereby incorporated by reference in its entirety). Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

The term “acid” contemplates all pharmaceutically acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically acceptable aliphatic, alicyclic and aromatic carboxylic acids, dicarboxylic acids, tricarboxylic acids, and fatty acids. Preferred acids are straight chain or branched, saturated or unsaturated C1-C20 aliphatic carboxylic acids, which are optionally substituted by halogen or by hydroxyl groups, or C6-C12 aromatic carboxylic acids. Examples of such acids are carbonic acid, formic acid, fumaric acid, acetic acid, propionic acid, isopropionic acid, valeric acid, alpha-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tataric acid and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of PCS, or one or more symptoms thereof, prevent the advancement of conditions or symptoms related to PCS, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002); each of which hereby incorporated by reference in its entirety).

The terms “animal,” “subject” and “patient” as used herein include all members of the animal kingdom including, but not limited to, mammals (e.g., mice, rats, cats, monkeys, dogs, horses, swine, etc.) and humans.

Concussion refers to an immediate and transient loss or perturbation of consciousness accompanied by a brief period of amnesia after a blow to the head (J. Head Trauma Rehabil. 2010, 25, 72-80; J. Head Trauma Rehabil. 2008, 23, 84-91; European Neurology 2007, 59, 113-119; J. Head Trauma Rehabil. 2006, 21, 375-378; New Eng. J. Med. 2007, 356, 166-172); each of which hereby incorporated by reference in its entirety). It is also the most common injury to American combat personnel (J. Head Trauma Rehabil. 2010, 25, 307-312; J. Head Trauma Rehabil. 2010, 25, 1-8; J. Psychosoc. Nurs. Ment. Health Serv. 2010, 48, 22-28; Am. J. Epidemiol. 2008, 167, 1446-1452; each of which hereby incorporated by reference in its entirety).

The pathophysiology of concussion is unknown. Suggested mechanisms include temporary disruption of the reticular activating system, self-limiting seizure activity, transient changes in intracranial pressure, release of inhibitory neurotransmitters, and perturbations of local ion concentrations and metabolic rates, all resulting from sudden rotational forces on the cerebral hemispheres (N. Eng. J. Med, 2007, 356(2), 166-172; Prog. Neurobiol. 2002, 67(4), 281-344; Curr. Opin. Psychiatry 2005, 18(3), 301-317; each of which hereby incorporated by reference in its entirety). It is also unknown why a subset of patients with concussion go on to have PCS. Even the epidemiology of PCS is unclear, with studies reporting rates ranging from 1-5% of diagnosed cases of concussion to 44% (Brain Inj. 2009, 23(3), 213-219; Curr. Opin. Psychiatry 2005, 18(3), 301-317; McCrea, M. A. in Mild Traumatic Brain Injury and Postconcussion Syndrome. New York: Oxford Univ. Press (2008); each of which hereby incorporated by reference in its entirety). Reasons cited for this discrepancy include the vague and subjective nature of the diagnostic criteria, overlap of PCS symptoms with those of post-traumatic stress disorder, depression, substance abuse, and the reinforcement of symptoms and “invalidism” by litigation (Curr. Opin. Psychiatry 2005, 18(3), 301-317; Brain Inj. 1999, 13(3), 151-172; N. Engl. J. Med. 2008, 358(5), 525-527; Arch. Clin. Neuropsychol. 2006, 21(4), 303-310; Arch. Phys. Med. Rehabil. 2009, 90(7), 1084-1093; each of which hereby incorporated by reference in its entirety). Traditionally, PCS has been regarded as being more of a “psychosomatic” condition than a reflection of neuronal damage—the ICD-10 criteria cite “subjective memory difficulties without neuropsychological evidence of marked impairment . . . hypochondriacal concerns and adoption of sick role” (See, World Health Organization. The ICD-10 Classification of Mental and Behavioral Disorders: Diagnostic Criteria for Research. Geneva: World Health Organization, 1993; hereby incorporated by reference in its entirety).

Another difficulty in the diagnosis and understanding of PCS is the fact that, thus far, the putative neurological injury is below the threshold of detection of clinically available imaging technology, and not consistently or usefully within the scope of newer techniques such as diffusion tensor imaging and magnetoencephlography (Brain 2009, 132(pt. 10), 2850-2870; J. Neurotrauma 2009, 26, 1213-1226; Neurology 2008, 70(12), 948-955; each of which hereby incorporated by reference in its entirety). Post-traumatic vertigo is also a common symptom of PCS. Limited studies have demonstrated often intractable nature of post-traumatic vertigo (Eur. Spine J. 2005, 15(6), 886-890; Laryngoscope 2004, 114(10), 1720-1723; each of which hereby incorporated by reference in its entirety). Heitger and colleagues have presented data showing that impaired eye movements are significantly correlated with PCS symptoms when such factors as depression, malingering, and intellectual ability are controlled for (Brain 2009, 132(pt. 10), 2850-2870; hereby incorporated by reference in its entirety). This supports prior studies that have suggested measurable deficits in motor control and eye movement function in PCS (Brain 2006, 129(Pt 3), 747-753; Brain 2004, 127(Pt 3), 575-590; Br. J. Sports Med. 2007, 41(9), 610-612; Neurosci. Lett. 2006, 401(1-2), 108-113; each of which hereby incorporated by reference in its entirety).

Downbeat nystagmus is also commonly found in patients with type-2 episodic ataxia (EA2), an autosomal dominant hereditary disorder usually manifested early in life that is characterized by hours' long bouts of severe ataxia triggered by physical or emotional stress (Brain 2007, 130(Pt 10), 2484-2493; hereby incorporated by reference in its entirety). The disease locus of EA2 was mapped to the calcium channel gene CACNA1A on chromosome 19p, which encodes the Caᵥ 2.1 subunit of the PQ calcium channel expressed mainly on cerebellar Purkinje cells (Curr. Opin. Neurol. 2006, 19(1), 33-40; Hum. Mol. Genet. 1997, 6(11), 1973-1978; Cell 1996, 87(3), 543-552; Ann. Neurol. 1995, 37(3), 289-293; each of which hereby incorporated by reference in its entirety). EA2 symptoms were serendipitously found to be attenuated by acetazolamide (Curr. Opin. Neurol. 2006, 19(1), 33-40; Ann. Neurol. 1995, 37(3), 285-287; each of which hereby incorporated by reference in its entirety). Strupp and colleagues have reported that symptoms of EA2 (including downbeat nystagmus) are both relieved and prevented by 4AP, presumably by improving cerebellar Purkinje cell functioning (Curr. Opin. Neurol. 2006, 19(1), 33-40; Prog. Brain Res. 2008, 171, 535-541; Expert Opin. Pharmacother. 2009, 10(10), 1537-1548; Neurology 2004, 62(9), 1623-1625; J. Neuroscience 2005, 25, 4141-4145; each of which hereby incorporated by reference in its entirety). We are not aware of any reports of EA2 being caused or precipitated by brain injury, particularly traumatic brain injury.

4-Amino pyridine (4-AP or 4AP) is a voltage-gated fast potassium-channel blocking agent that has been shown to improve conduction in demyelinated axons (J. Physiol. 1981, 313, 301-315; Nature 1980, 283(5747), 570-572; Neuroscience 1997, 77(2), 553-562; each of which hereby incorporated by reference in its entirety). 4AP improves functioning of damaged cerebellar Purkinje cells, which provide tonic GABA-mediated inhibition of vestibular nuclei (Brain 2005, 128(Pt 6), 1237-1246; Exp. Brain Res. 2001, 129(4), 419-425; each of which hereby incorporated by reference in its entirety). There have been relatively few studies of cerebellar damage after traumatic brain injury (Prog. Brain Res. 2007, 161, 327-338; hereby incorporated by reference in its entirety). However, there have been reports of surprising cerebellar atrophy in brain injured children who do not have evidence of direct damage to the cerebellum (Childs Nerv. Syst. 2001, 17(4-5), 263-269; Am. J. Neuroradiol. 2007, 28(3), 537-542; each of which hereby incorporated by reference in its entirety). Recent animal studies have demonstrated the unique vulnerability of cerebellar Purkinje cells to percussive injury; however the connection between such is poorly understood (Prog. Brain Res. 2007, 161, 327-338; Cerebellum 2009, 8(3), 211-221; each of which hereby incorporated by reference in its entirety). Flocullonodular Purkinje cells are also vulnerable to toxic, anoxic, and ischemic injury. More recent studies have shown that, in contrast to what is commonly believed, 4-AP does not increase Purkinje cell excitability, spontaneous activity or firing rate in models of episodic ataxia, nor does it alter synaptic transmission (J. Neurosci. 2010, 30, 7258-7268; herein incorporated by reference in its entirety). Rather, the only discernable functional consequence of treatment with 4-AP was restoration of the precision of pacemaking in EA2 mutant Purkinje cells via blockage of potassium channels.

Post-concussive symptoms such as vertigo, disequilibrium, difficulty with static or mobile vision, and difficulty with integrating multiple stimuli may in at least some cases represent impaired functioning of cerebellar Purkinje cells or their axons. Surprisingly, according to the present invention, it has been found that such injury, as well as other defects of Purkinje cell output, may be ameliorated by 4-AP.

Herein, studies of a subject with PCS are presented that indicate improvement of symptoms and post-traumatic impairments of eye movement upon administration of a compound that blocks voltage-gated potassium-channels, such as 4-aminopyridine.

In experimental and clinical settings, 4-AP has been shown to improve conduction in demyelinated axons (See, U.S. Pat. Nos. 5,370,879; 5,540,938; each incorporated herein by reference in its entirety). 4-AP has been demonstrated to be a useful agent in treating several nervous system disorders, including multiple sclerosis (See, for example, Exp Neurol. 2011, 227(1), 232-235; J. Vet. Pharmacol. Ther. 2009, 32(5), 485-491; J. Emerg. Med. 2006, 30(2), 175-177; each of which hereby incorporated by reference in its entirety). The FDA has approved an extended release form of 4-AP, marketed by Acorda Therapeutics, Inc. under the trade name Ampyra®, for use in patients with multiple sclerosis. In clinical studies, orally administered 4-AP demonstrated mild to moderate improvements in restoring nerve impulse transmission in multiple sclerosis patients over a period of days. 4-AP is also discussed in, for example, U.S. Pat. Nos. 5,545,648; 4,880,819; and 4,508,715; and U.S. Patent Publication No. 20050059709; each of which are incorporated herein by reference in its entirety. Other potassium channel blockers are described, for instance, in U.S. Patent Publication Nos. 20100092569; 20100016444; and 20040157889; each of which are incorporated herein by reference in its entirety.

4AP has also been shown to relieve downbeat nystagmus (DBN) (Neurology 2003, 61(2), 165-170; Curr. Opin. Neurol. 2006, 19(1), 33-40; Prog. Brain Res. 2008, 171, 535-541; Brain 2007, 130 (Pt 9), 2441-2451; each of which hereby incorporated by reference in its entirety). DBN is a centrally mediated vestibular disorder usually associated with chronic oscillopsia and postural instability. Cerebellar ischemia and degeneration are cited as the most common etiologies of DBN (Brain 2005, 128(Pt 6), 1237-1246; J. Neurol. Neurosurg. Psychiatry 2008, 79(6), 672-677; each of which hereby incorporated by reference in its entirety). A mechanism of action of 4AP in DBN has been described (Neurology 2003, 61(2), 165-170; Neurology 2003, 61(2), 158-159; each of which hereby incorporated by reference in its entirety). GABA-ergic efferents from cerebellar Purkinje cells project to and inhibit anterior semicircular canal projections to vestibular nuclei. Impairment of this inhibition causes upward gaze drift and consequent downbeat nystagmus. Functional MRI studies have corroborated floccular hypofunction in patients with idiopathic downbeat nystagmus (Neurology 2007, 69(11), 1128-1135; Neurology 2006, 66(2), 281-283; each of which hereby incorporated by reference in its entirety). 4AP has been shown to increase the excitability of cerebellar Purkinje cells in vitro (Exp. Brain Res. 2001, 139(4), 419-425; hereby incorporated by reference in its entirety). This increase in Purkinje cell excitability may improve functioning in damaged cells, restoring normal inhibition of vestibular nuclei (Prog. Brain Res. 2008, 171, 535-541; Brain 2007, 130 (Pt 9), 2441-2451; each of which hereby incorporated by reference in its entirety).

Less evidence exists demonstrating that 4-AP or related compounds may be effective in treating symptoms associated with other chronic neurological disorders. In a 2004 study in patients with idiopathic cerebellar ataxia, the potassium channel blocker 3,4-diamino pyridine (“3,4-DAP”) was shown to be effective in ameliorating some gravity-dependent symptoms associated with ataxic episodes (Sprenger, A., et al. Neurol. 2006, 67: 905-07, incorporated herein by reference in its entirety). 4-AP has been also used to treat patients diagnosed with type-2 episodic ataxia (EA2), a relatively rare hereditary disorder usually manifested early in life characterized by severe ataxia triggered by physical or emotional stress (Strupp, M., et al. Neurol. 2004, 62, 1623-25, incorporated herein by reference in its entirety). EA2 is now known to relate to mutations of the calcium channel gene CACNA1A encoding the Caᵥ2.1 subunit of the P/Q-type calcium channel highly expressed on the surface of Purkinje cells of the cerebellum. Other than the foregoing, the use of 4-AP in treating neurological disorders has been somewhat limited.

Treatment of PCS presents another set of challenges. To the extent any consensus view exists among experts, the common understanding is that PCS is an amorphous, non-specific medical condition that are neither singular in type nor uniform in presentation or response to treatment (See, Current Treatment Options in Neurology 2000, 2:169-186; J. Neuropsychiatry Clin. Neurosci. 2009, 21(2), 181-188; each of which hereby incorporated by reference in its entirety). PCS symptoms do not appear to cluster together in an invariate or even consistently predictable fashion like other neurological disorders and deficits. This lack of cohesive symptomology fails to support the existence of any coupled response to pharmacological or physical intervention. Instead, multiple and varied treatments are generally required for the multiple and varied symptoms of these individuals (See Arciniegas, et al. Neuropsych. Dis. Treat. 2005, 1, 311-327; hereby incorporated by reference in its entirety). Generally speaking, one cannot point to a single point of physical injury that underlies PCS, and likewise, cannot suggest that any of its varied symptoms may be amenable to a single medication.

Other authorities abandon physical causes of PCS entirely, and focus instead on a purported emotional cause for PCS. Rather than advocating pharmacological intervention, these approaches stress patient education, social intervention, and psychotherapy as primary treatments. Bryant (New Engl. J. Med. 2008, 358, 525; hereby incorporated by reference in its entirety) notes “psychological factors account for many postconcussive symptoms [and] suggests that more effective interventions may involve augmenting educational programs with strategies that aim to reduce [post traumatic stress disorder] and depression” (See also, McRea (2008) Mild Traumatic Brain Injury and Postconcussion Syndrome, Oxford University Press; Iverson et al. Arch. Clin. Neuropsychol. 2006, 21,303-310; each of which hereby incorporated by reference in its entirety).

While treatment with 4-AP has been attempted to resolve some neurological afflictions, there have been no reports of potential utility in the treatment of a condition such as PCS. The non-specific nature of the mechanism of action of 4-AP, as well as the non-specific nature of PCS, precluded interest by experts or professionals as evidenced, for example, by the absence of reports in the literature to date for such treatment. As described in the literature, 4-AP may rectify the functioning of calcium channel deficiencies found in rare genetic disorders of the CACNA1A gene, which is expressed in Purkinje cells of the cerebellum. The concept or possibility that 4-AP may augment or improve Purkinje cell functioning in the absence of specific and clearly defined genetic disorders has not been described. An addition, the possibility that the clusters of incapacitating symptoms of PCS may be related to defects in the functioning of Purkinje cells has not been noted. In contrast, the inventors have shown marked symptomatic improvement using 4-AP when treating a subject having no abnormality of the CACNA1A gene.

Standard practices employed by those in the art should be followed to determine a therapeutically effective dose of 4-AP or a similar compound for each patient sufficient to produce beneficial results. For example, once a suitably low starting dose is chosen, for example, 1 or 5 or 10 mg per day, the subject may remain under the observation of a trained health professional for about 2 hours after administration, both to observe for side effects and to provide appropriate care and reassurance should any adverse reactions occur. Barring significant adverse reactions, the subject may continue to take that low dose for about 72 hours prior to assessment of effects of the medication. With appropriate observation and supervision, the dose can also be increased to obtain an improvement in results. Therapeutically effective dosages of orally administered 4-AP for treatment of the identified subset of PCS patient described herein are preferably between about 1 to about 100 milligrams per day, more preferably between about 5 to about 30 milligrams per day, and most preferably between about 10 to about 20 milligrams per day.

The capacity of a nonspecific agent like 4-AP to ameliorate any symptoms exhibited by PCS patients was completely unexpected and inconsistent with the current understanding and teachings of those of skill in the art. Even more unexpected was the ability of 4-AP to elicit improvements in a diverse variety of incapacitating symptoms. By way of non-limiting example, neuro-optometric and neurofeedback evaluations indicated dramatic and unexpected improvements in symptoms related to, for example, balance impairments, vertigo, cognitive impairments, emotional impairments, somatosensory impairments, neurosynchronization, and restoration of brain activity.

The Rivermead Post-Concussion Symptoms Questionaire (RPSCQ), which groups symptoms into three groups (mood and cognition symptoms, general somatic symptoms, and visual somatic symptoms) is often used to assess physicians in a diagnosis of PCS (J. Neuropsychiatry Clin. Neurosci. 2009, 21, 181-188; hereby incorporated by reference in its entirety). Both the International Statistical Classification of Diseases and Related Health Problems, 10th Rev. (“ICD-10”) and Diagnostic and Statistical Manual of Mental Disorders, 4th Ed. (“DSM-IV”), each of which hereby incorporated by reference in its entirety, include further provisional diagnostic criteria for PCS. However, one difficulty with the Rivermead analysis is that the questionnaire is based on self-reported responses from the subject. While the Rivermead analysis is useful in determining the presence of PCS, the results obtained are subjective. The ICD-10 describes postconcussional syndrome as occurring following head trauma (usually sufficient to result on loss of consciousness) and recommends that the subject exhibit at least three symptoms from six categories as follows: 1) headache, dizziness, chronic fatigue, or noise intolerance; 2) emotional lability, irritability, depression, or anxiety; 3) concentration, memory or intellectual difficulties without neuropsychological evidence of marked impairment; 4) insomnia; 5) reduced alcohol tolerance; and 6) preoccupation with the preceeding symptoms and fear of permanent brain damage, with hypochondriacal concern and adoption of sick role (New Eng. J. Med. 2007, 356, 166-172; hereby incorporated by reference in its entirety). However, the symptoms are not weighted in the analytic framework; nor, for example, are balance impairments, vertigo, or somatosensory impairments categorized separately. The DSM-IV diagnostic criteria also does not separately categorize, for example, vertigo and balance impairments, and recommends that the subject exhibit at least three symptoms from a group of categories without further weighting the symptoms.

In one embodiment, a diagnostic method for identifying, classifying or diagnosing a subject suffering from PCS is presented, wherein symptoms are grouped into symptom clusters based on symptom class. In one embodiment, the method comprises evaluating a subject for exhibition of symptoms in one or more symptom clusters and categorizing symptoms exhibited by said subject into one or more symptom classes. In one embodiment, the symptom classes comprise symptoms related to balance impairments, symptoms related to vertigo, symptoms related to cognitive or emotional impairments, and symptoms related to somatosensory impairments. In yet another embodiment, the methods comprise administering to said subject a therapeutically effective amount of a potassium channel blocker.

In one embodiment, the subject has a history of head trauma, with or without loss of consciousness. In some embodiments, the head trauma occurred about 48 hours prior to evaluation; from about 48 hours to about 1 week prior to evaluation; from about 1 week to about 2 weeks prior to evaluation; from about 2 weeks to about 3 weeks prior to evaluation; from about 3 weeks to about 4 weeks prior to evaluation; from about 4 weeks to about 5 weeks prior to evaluation; from about 5 weeks to about 6 weeks prior to evaluation; from about 6 weeks to about 7 weeks prior to evaluation; from about 7 weeks to about 8 weeks prior to evaluation; from about 8 weeks to about 9 weeks prior to evaluation; from about 9 weeks to about 10 weeks prior to evaluation; from about 10 weeks to about 11 weeks prior to evaluation; from about 11 weeks to about 12 weeks prior to evaluation; or more than about 12 weeks prior to evaluation. It will be recognized that any of the times listed herein may constitute an upper or lower time range, and may be combined with any other time range to constitute a time range comprising an upper or lower limit.

In one embodiment, symptoms are intermittent. In one embodiment, the symptoms are chronic. In one embodiment, symptoms last more than about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, or more than 16 weeks. It will be recognized that any of the times listed herein may constitute an upper or lower time range, and may be combined with any other time range to constitute a time range comprising an upper or lower limit.

In one embodiment, the methods comprise determining that the subject exhibits symptoms selected from at least two system clusters selected from the group consisting of symptoms related to balance impairments, symptoms related to vertigo, symptoms related to cognitive or emotional impairments, and symptoms related to somatosensory impairments.

In one embodiment, the subject exhibits symptoms from at least three symptom clusters selected from the group consisting of symptoms related to balance impairments, symptoms related to vertigo, symptoms related to cognitive or emotional impairments, and symptoms related to somatosensory impairments.

In one embodiment, the subject exhibits at least one symptom from each of the following symptom clusters: symptoms related to balance impairments, symptoms related to vertigo, symptoms related to cognitive or emotional impairments, and symptoms related to somatosensory impairments.

In one embodiment, the subject is evaluated with respect to a framework of four broad symptom clusters, A-D, according to Table 1. Table 1 exhibits exemplary conditions and/or presentations for each symptom class.

TABLE 1

| | Symptom Class | Examples |
|---|---|---|
| Cluster A | Balance | Static or dynamic balance difficulties, |
| | Impairments | including problems maintaining |
| | | balance while off-center or distracted, |
| | | reaching, or walking |
| Cluster B | Vertigo | Episodic failures in balance, including |
| | | vestibular seizures and oscillopsia, |
| | | presence of persistent sensations of |
| | | motion or constant tilt |
| Cluster C | Cognitive or | An inability to process newly presented |
| | Emotional | information, inability to tolerate |
| | Impairments | distraction, slowing of cognitive |
| | | functions, emotional lability, apathy, |
| | | irritability |
| Cluster D | Somatosensory | Impairments to oculomotor function, |
| | Impairments | intolerance of noise, light or |
| | | commotion, anergy, chronic fatigue, |
| | | hypersomnia, headache |

In one embodiment, the symptoms related to balance impairments are selected from the group consisting of difficulty in standing for more than 5 minutes, inability to maintain stability while standing with eyes closed or with feet together, impairment of gait on a smooth surface, impairment of gait on an uneven surface, difficulty moving body position, difficulty rising unaided from a sitting position or from sitting on a floor, difficulty in reaching forward or overhead with an outstretched arm while standing, inability to maintain heel-to-toe gait, difficulty maintaining balance while looking over one shoulder, difficulty conversing while walking, and difficulty tossing objects from hand to hand while walking.

In one embodiment, the symptoms related to vertigo are selected from the group consisting of vertigo, persistent sensation of being off-balance or tilted, persistent sensation of walking on soft or moving surfaces, persistence or perseveration of proprioceptive sensations, oscillopsia, and vestibular seizures.

In one embodiment, the symptoms related to cognitive or emotional impairments are selected from the group consisting of persistent difficulty with registration or retention of newly presented information in any modality, distractibility, inability to tolerate distraction, impairment of judgment, inability to perform two simple tasks concurrently, slowed speed of mental processing or responding, emotional lability or exaggerated emotions, persistent or frequent irritability, and apathy, listlessness, or blunting of affect.

In one embodiment, the symptoms related to somatosensory impairments are selected from the group consisting of headache, insomnia, anergy, chronic fatigue or hypersomnia, difficulty in accommodation or focus, diplopia, nystagmus, impairment of tracking an object in motion, and intolerance of noise, light or commotion.

In some embodiments, the method may be further refined to more precisely define the subject class or aid in designation of inclusion criteria for clinical evaluations of new therapeutic interventions.

In one embodiment, a point system is presented in which symptoms from each cluster are further assigned a point value. In one embodiment, the point system is presented as shown in Table 2. The evaluation should rate the frequency of each symptom over the span of about one week prior to the assessment, according to the following scale: 0—Never present, 1—Sometimes present, 2—Often present, 3—Always or Nearly always present. In this context, occurrence of a symptom from about 1 to 5 times per week would classify as sometimes present, occurrence from about 5 to about 10 times per week would classify as often present, and occurrence about 10 or more times per week would classify as always or nearly always present. The rating number for each item should be recorded as the raw score. Each raw score should then be multiplied by the weight factor to obtain the weighted score.

TABLE 2
PCS Symptom Cluster Scale
PCS SYMPTOM CLUSTER SCALE

| | | | |
|---|---|---|---|
| | Raw | | Weighted |
| Symptom | score | Weight | Score |

| | | |
|---|---|---|
| Cluster A | Difficulty in standing for more than 5 minutes | 2 |
| | Inability to maintain stability while standing with | 3 |
| | eyes closed or with feet together | |
| | Impairment of gait | 3 |
| | Difficulty moving body position (e.g., from chair to | 1 |
| | bed or toilet) | |
| | Difficulty rising unaided from a sitting position or | 1 |
| | from sitting on the floor | |
| | Inability to maintain heel-to-toe gait | 1 |
| | Difficulty maintaining balance while looking over | 2 |
| | shoulder | |
| | Difficulty conversing while walking | 3 |
| | Difficulty tossing objects from hand to hand while | 1 |
| | walking | |
| Cluster B | Vertigo | 2 |
| | Persistent sensation of being off-balance or tilted | 4 |
| | Persistent sensation of walking on soft or moving | 3 |
| | surface | |
| | Persistence or perseveration of proprioceptive | 4 |
| | sensations (e.g., feeling motion after coming to rest) | |
| | Oscillopsia (perception of stationary objects | 4 |
| | moving) | |
| | Vestibular seizures | 5 |
| Cluster C | Persisting difficulty with registration or retention of | 2 |
| | newly presented information in any modality | |
| | Inability to tolerate distraction (e.g., inability to | 3 |
| | complete simple task or resume conversation after | |
| | interruption) | |
| | Inability to perform two simple tasks concurrently | 3 |
| | (“multitasking”) | |
| | Slowed speed of mental processing or responding | 2 |
| | Emotional lability or exaggerated emotions | 2 |
| | Persistent or frequent irritability | 2 |
| | Apathy, listlessness, blunting of affect | 2 |
| | Intolerance of noise, light, or commotion | 4 |
| Cluster D | Headache | 2 |
| | Insomnia | 1 |
| | Anergy, chronic fatigue, hypersomnia | 3 |
| | Difficulty in accommodation or focus | 3 |
| | Diplopia | 2 |
| | Nystagmus | 4 |
| | Impairment of tracking an object in motion | 4 |

In some embodiments, the frequency of each symptom is rated over the course of about three days. In some embodiments, the frequency of each symptom is rated over the course of about four days. In some embodiments, the frequency of each symptom is rated over the course of about five days. In some embodiments, the frequency of each symptom is rated over the course of about six days. In some embodiments, the frequency of each symptom is rated over the course of about one week. In some embodiments, the frequency is rated over the course of about 10 days. In some embodiments, the frequency is rated over the course of about two weeks. In some embodiments, the frequency is rated over the course of about three weeks. In some embodiments, the frequency is rated over the course of about four weeks. In some embodiments, the frequency is rated over the course of more than about four weeks. It will be recognized that any time period listed herein may constitute an upper or lower time limit, and may be combined with any other time period to constitute a time period comprising an upper and lower limit.

In one embodiment, the methods comprise evaluating a patient and assigning point values to the symptoms presented. In one embodiment, the point totals are weighted according to symptom. In one embodiment, the point values are assigned as designated in Table 2. In one embodiment, the subject exhibiting a point total of about 50 or greater will be likely to benefit from treatment for PCS, wherein the point total is distributed between at least two symptom clusters. In one embodiment, the subject exhibiting a point total of about 100 or greater will be a candidate for treatment for PCS. In some embodiments, the subject exhibits a point total of from about 40 to about 200. In some embodiments, the subject exhibits a point total of about 40, 50, 60, 70, 80, 90, 100, 110, 120, 130, 140, 150, 160, 170, 180, 190, 200, or greater than 200. It will be recognized that any point total listed herein may constitute an upper or lower point total, and may be combined with any other point total to constitute a point total range comprising an upper and lower limit. It will also be recognized that any point total or point range listed herein may indicate a likely benefit or response from treatment for PCS.

In one embodiment, the method may comprise assessment of the benefit of or response to treatment, for example 4-AP, for PCS following administration of said treatment. In one embodiment, assessment of the benefit or response comprises evaluating the subject and assigning point values to the symptoms presented. In one embodiment, the point totals are weighted according to symptom. In one embodiment, reduction of point total exhibited by a subject relative to the point total scored by the subject prior to initiation of treatment is measured. In one embodiment, reduction of point total exhibited by a subject relative to the point total scored by the subject in a previous evaluation is measured. In one embodiment, the point total is reduced by from about 10% to about 90%. In one embodiment, the point total is reduced by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater than about 95%. It will be recognized that any percent listed herein may constitute an upper or lower percent, and may be combined with any other percent to constitute a percent range comprising an upper and lower limit.

This point system may also be used to assess the benefit of treatment with 4-AP and similar pharmacological agents. For example, a reduction in the overall number of scored points can be an indicator of efficacy, and in this context, a greater than, for example, 50% reduction in point number may be considered a significant response.

In another embodiment, the method of treatment comprises the steps of: i) identifying, classifying and/or diagnosing a subject in need of such treatment; (ii) providing a compound that modulates neuronal ion channel function, or a pharmaceutically acceptable salt, hydrate, or solvate thereof; and (iii) administering said compound in a therapeutically effective amount to a subject in need of such treatment. In one embodiment, the method further comprises (iv) assessment of the benefit of or response to treatment.

In another embodiment, the method of treatment comprises the steps of: i) identifying, classifying and/or diagnosing a subject in need of such treatment; (ii) providing a composition comprising a compound that modulates neuronal ion channel function, or a pharmaceutically acceptable salt, hydrate, or solvate thereof; and (iii) administering said composition in a therapeutically effective amount to a subject in need of such treatment. In one embodiment, the method further comprises (iv) assessment of the benefit of or response to treatment.

In one embodiment, identifying, classifying, diagnosing, evaluating and/or assessing may comprise administration of a neurological, neuro-optometric, psychological, physical and/or psychiatric examination to the subject. In one embodiment, identifying, classifying, diagnosing, evaluating and/or assessing may be performed by a physician. In one embodiment, evaluation is neurological. In one embodiment, evaluation is neuro-optometric. In one embodiment, evaluation is neuro-otological. In one embodiment, evaluation is neuro-physiological. In one embodiment, evaluation is neuro-psychological. In one embodiment, evaluation is neuro-ophthamologicial. In one embodiment, evaluation is psychological. In one embodiment, evaluation is physical. In one embodiment, evaluation is psychiatric.

In one embodiment, the methods comprise administering to the subject an effective amount of a compound that modulates neuronal ion channel function, or a pharmaceutically acceptable salt, solvate, or hydrate; or a composition comprising a compound that modulates neuronal ion channel function, or a pharmaceutically acceptable salt, solvate, or hydrate, and a pharmaceutically acceptable carrier. The pharmaceutically acceptable carriers are well-known to those skilled in the art, and include, for example, adjuvants, diluents, excipients, fillers, lubricants and vehicles. Often, the pharmaceutically acceptable carrier is chemically inert toward the active compounds and is non-toxic under the conditions of use. Examples of pharmaceutically acceptable carriers may include, for example, water or saline solution, polymers such as polyethylene glycol, carbohydrates and derivatives thereof, oils, fatty acids, or alcohols.

In one embodiment, the compounds of the invention are formulated into pharmaceutical compositions for administration to subjects in a biologically compatible form suitable for administration in vivo. According to another aspect, the present invention provides a pharmaceutical composition comprising a compound in admixture with a pharmaceutically acceptable diluent and/or carrier. The pharmaceutically-acceptable carrier is “acceptable” in the sense of being compatible with the other ingredients of the composition and not deleterious to the recipient thereof. The pharmaceutically-acceptable carriers employed herein may be selected from various organic or inorganic materials that are used as materials for pharmaceutical formulations and which are incorporated as absorption delaying agents, analgesics, antibacterials, antifungals, buffers, binders, coatings, disintegrants, diluents, dispersants, emulsifiers, excipients, extenders, glidants, solubilizers, solvents, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In one embodiment, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine,

When administered to a subject, the compound of the invention and pharmaceutically acceptable carriers can be sterile. Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain minor amounts of wetting or emulsifying agents, or pH buffering agents.

The pharmaceutical formulations of the present invention are prepared by methods well-known in the pharmaceutical arts. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

Additionally, the compounds and/or compositions of the present invention are administered to a human or animal subject by known procedures including oral administration, intraperitoneal, parenteral (e.g., intravenous), intradermal, subcutaneous, intranasal, transdermal, topical, transmucosal, rectal, sublingual or buccal administration. In one preferred embodiment, the agent is administered orally.

For oral administration, a formulation of the compounds of the invention may be presented in dosage forms such as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, for example, sweetening agents such as fructose, aspartame or saccharin; flavoring agents such as peppermint, oil of wintergreen, or cherry; coloring agents; and preservative agents, to provide a pharmaceutically palatable preparation.

The compositions may further comprise one or more sterile diluents, such as water, saline solutions, fixed oils, polyalkylene glycols, polyoxyalkylene glycols, glycerine, or other solvents; antibacterial agents such as benzyl alcohol or methyl parabens, antioxidants such as ascorbic acid, citric acid or sodium bisulfite, chelating agents such as EDTA, buffers such as acetate, citrate, phosphate and the like, tonicity adjusters such as sodium chloride or dextrose, pH adjusters such as weak acids or bases, etc.

In some embodiments, the composition is in unit dose form such as a tablet, capsule or single-dose vial. Suitable unit doses, i.e., therapeutically effective amounts, may be determined during clinical trials designed appropriately for each of the conditions for which administration of a chosen compound is indicated and will, of course, vary depending on the desired clinical endpoint.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

An exemplary form of 4-AP composition is an extended release form of 4-AP, under the trade name Ampyra®. Thus, in one embodiment, the methods comprise administration of an extended release form of 4-AP. In one embodiment, the extended release form of 4-AP is Amypra®.

Systemic administration can also be via transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In accordance with the methods of the present invention, the compounds of the invention are administered to the subject in a therapeutically effective amount, for example to reduce or ameliorate symptoms related to PCS in the subject. This amount is readily determined by the skilled artisan, based upon known procedures, including analysis of titration curves established in vivo and methods and assays disclosed herein.

In one embodiment, the methods comprise administration of a therapeutically effective dosage of the compounds of the invention. In some embodiments, the therapeutically effective dosage is at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the methods comprise a single dosage or administration (e.g., as a single injection or deposition). Alternatively, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

The precise dose to be employed in the compositions will also depend on the route of administration, and should be decided according to the judgment of the practitioner and each patient's circumstances. In specific embodiments of the invention, suitable dose ranges for oral administration of the compounds of the invention are generally about 1 mg/day to about 1000 mg/day. In one embodiment, the oral dose is about 1 mg/day to about 800 mg/day. In one embodiment, the oral dose is about 1 mg/day to about 500 mg/day. In another embodiment, the oral dose is about 1 mg/day to about 250 mg/day. In another embodiment, the oral dose is about 1 mg/day to about 100 mg/day. In another embodiment, the oral dose is about 5 mg/day to about 50 mg/day. In another embodiment, the oral dose is about 5 mg/day. In another embodiment, the oral dose is about 10 mg/day. In another embodiment, the oral dose is about 20 mg/day. In another embodiment, the oral dose is about 30 mg/day. In another embodiment, the oral dose is about 40 mg/day. In another embodiment, the oral dose is about 50 mg/day. In another embodiment, the oral dose is about 60 mg/day. In another embodiment, the oral dose is about 70 mg/day. In another embodiment, the oral dose is about 100 mg/day. It will be recognized that any of the dosages listed herein may constitute an upper or lower dosage range, and may be combined with any other dosage to constitute a dosage range comprising an upper and lower limit.

Any of the compounds and/or compositions of the invention may be provided in a kit comprising the compounds and/or compositions. The kit may be therapeutic kit to treat, suppress and/or prevent concussive disorders, and/or a kit to identify, classify and/or diagnose concussive disorders. In one embodiment, the compound and/or composition of the invention is provided in a kit. In a specific embodiment, 4-AP is provided in a kit.

The components of the kit may comprise a list of symptom clusters. In one embodiment, the components of the kit comprise a list of symptom clusters selected from the group consisting of balance impairments, vertigo, cognitive or emotional impairments, and somatosensory impairments. In one embodiment, the symptom clusters are classified according to table 1 herein. In another embodiment, the symptom clusters are further classified according to table 2 herein. In one embodiment, the kit may comprise instructions to a physician for identifying, classifying, diagnosing, or evaluating a subject according to the methods described herein. In one embodiment, the kit may comprise instructions to a physician for treating a subject according to the methods described herein.

In one embodiment, the methods comprise a kit. In one embodiment, the methods comprise providing a kit to the subject. In one embodiment, the methods comprise providing a kit to a physician. In one embodiment, the methods comprise a kit for identifying, classifying, diagnosing, treating, evaluating or assessing a subject.

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

Examples are provided below to facilitate a more complete understanding of the invention. The following examples serve to illustrate the exemplary modes of making and practicing the invention. However, the scope of the invention is not to be construed as limited to specific embodiments disclosed in these Examples, which are illustrative only.

### Example 1

### Case Study of a Patient with Incapacitating Post-Concussive Symptoms and Eye Movement Disorder Treated with 4-Aminopyridine

A 40 year old man (the “Subject”) with no prior neurological history was involved in a motor vehicle accident in which he sustained a traumatic brain injury (TBI). The Subject's airbag deployed and his car swerved off the road. Emergency personnel found the Subject conscious in his car shortly after the accident. The Subject recalls being dazed but has no recollection of losing or regaining consciousness. The Subject was immediately taken to a local emergency room, where, after examination including cervical X-rays, he was found to have no apparent injury and released home. The next day, the Subject returned to the emergency room complaining of intractable dizziness, head and neck pain, and forgetfulness. A CT scan of the Subject's head was normal, and the Subject was referred for further outpatient neurological treatment. The Subject underwent an Epley maneuver and received physical therapy for disequilibrium at a clinic specializing in TBI rehabilitation.

Despite months of therapy, the Subject's symptoms dizziness and disequilibrium continued unabated, with the Subject reporting intractable dizziness and disequilibrium, with a constant feeling as if he were sharply tilting to his right. In addition, the Subject experienced marked noise and light sensitivity, an inability to tolerate any distraction, inability to maintain a train of thought in the presence of any other conversation or noise. Attempting to even briefly read or watch television worsened bi-temporal and occipital headaches. Simple conversation or noise of any kind would also worsen temporal and occipital headaches.

The Subject also reported marked initial insomnia, anergy, frequent vivid nightmares, an inability to track moving objects (e.g., a ball thrown by his child), and persistence of proprioceptive sensations (e.g., difficulty walking or standing after disembarking from an exercise bicycle, feeling as if his legs were still pedaling). The Subject also reported severe neuromuscular deficits—bouts of abrupt muscle weakness, grimacing, slurring of speech, hypersalivation, and relative unresponsiveness linked to physical exertion, emotional stress, or noisy or tumultuous environments. Although these episodes would last 2 to 4 hours, they required that the Subject be assisted to bedrest, and would often be followed by exhaustion for up to 24 hours. These episodes occurred several times a week for two years after the injury, after which time Subject undertook a radical reduction in activities in an effort to prevent their reoccurrence. The symptoms remained unchanged after successful resolution of litigation related to the motor vehicle accident.

Given the paroxysmal nature of the subject's bouts of weakness, the Subject was referred to a university hospital epilepsy monitoring unit. No electrophysiological seizure activity was found. Significant impairment was found on repeated neuropsychological testing. Initial encoding and immediate recall were between the 5^{th} and 37^{th} percentile, which is at or greater than one standard deviation below the Subject's expected values. Tests of delayed recognition recall were at the 9^{th} percentile, which also represents significant impairment, given the Subject's educational and occupational history. Neuro-optometric exams showed consistent convergence excess of the high tonic type, consistent with Subject's difficulty reading or using a computer; daily frontal, temporal and occipital headaches with increasing intensity; photopsia with rare dizziness; increased light sensitivity; impaired depth perception; increased sensitivity to visual motion; and dizziness or disequilibrium in multiply stimulating visual environments. On neuro-otological exam, subject was found to have a 29% right vestibular hypofunction on caloric testing and decreased right vestibular evoked myogenic potential. On nystagmography, subject was noted to have downbeat nystagmus in the dark at the 20° left gaze position, with weak visual suppression of vestibular nystagmus. These were noted to be signs of central pathology. The subject underwent diffusion tensor magnetic resonance imaging with no significant findings noted. Other general symptoms included, for example, easy fatigability, severe increased sensitivity to sound, difficulty understanding as well as expressing speech, severe impairment of auditory discrimination, tinnitus, decreased concentration, memory deficits, poor coordination, sleep disturbances, difficulty handwriting and periodic seizure-like activity with over-stimulation of visual and vestibular systems.

Prior to this injury, the Subject was working successfully as a technical writer. He was married and a father. H is spouse described him as having been generally upbeat, witty, and having a variety of avocational interests. History of substance abuse was convincingly denied. There was no prior history of any difficulty with balance, coordination, or motor control—subject was an avid amateur athlete and an excellent dancer. Prior medical history was significant for an apparent concussion at age 7—subject reports having slipped on an icy playground and waking up in his bed. No medical follow up or sequelae are recalled. Subject also reports a history of well controlled asthma and a 2 year history of recurrent nephrolithiasis that resolved with the removal of a parathyroid adenoma 4 years prior to the accident. At the time of the accident, patient was taking levothyroxine 112 mcg. daily (taken consistently since parathyroid surgery) and fluticazone/salmetrol inhaler twice daily. He was on no other prescribed or over the counter medication.

Since the accident, the Subject had been tried on a variety of anti-epileptics, benzodiazepines, atypical antipsychotics, vestibular suppressants, muscle relaxants, antidepressant medications, psychotherapy, and neurofeedback training. These were all at best modestly helpful, with incapacitating symptoms persisting. He was unable to tolerate rehabilitative balance training or cognitive remediation. He has been unable to work at his profession or to provide any more than minimal assistance with domestic chores.

The study Subject had persisting stress-induced episodes of weakness and dysequilibrium, along with DBN. A crude phenomenological similarity of these symptoms to EA2 was noted. DNA sequencing of Subject's CACNA1A gene was done; no abnormality was found. Given intractable, persisting, and incapacitating nature of Subject's symptoms and the absence of any therapeutic alternatives, an empiric clinical trial of 4AP was agreed upon. Given subject's history of nephrolithiasis, it was felt that 4AP would be less likely to cause significant adverse reactions than acetazolamide. Subject provided written informed consent.

Based on these initial observations and the Subject's medical history, the Subject was evaluated with respect to a framework of four broad symptom clusters, A-D, according to Table 1.

TABLE 1

| | Symptom Class | Examples |
|---|---|---|
| Cluster A | Balance | Static or dynamic balance difficulties, |
| | Impairments | including problems maintaining |
| | | balance while off-center or distracted, |
| | | reaching, or walking |
| Cluster B | Vertigo | Episodic failures in balance, including |
| | | vestibular seizures and oscillopsia, |
| | | presence of persistent sensations of |
| | | motion or constant tilt |
| Cluster C | Cognitive or | An inability to process newly presented |
| | Emotional | information, inability to tolerate |
| | Impairments | distraction, slowing of cognitive |
| | | functions, emotional lability, apathy, |
| | | irritability |
| Cluster D | Somatosensory | Impairments to oculomotor function, |
| | Impairments | intolerance of noise, light or |
| | | commotion, anergy, chronic fatigue, |
| | | hypersomnia, headache |

Information was obtained by direct observation and examination, the use of specific neurophysiological tests, and questionnaires designed to evaluate the presence or absence of significant symptoms in each cluster. As a result of this examination and analysis, it was determined that the Subject exhibited symptoms in all four clusters, and was determined to be a suitable candidate for treatment with 4-AP.

The Subject was treated by oral administration of 4-AP, starting with a minimal dose of 5 mg. No visible adverse effects were observed or reported within 3 hours of administration. Increasing doses were administered—from 5 mg of 4-AP twice per day, to 10 mg per day, to 30 mg per day, at which point the Subject reported a dramatic improvement in symptoms which lasted for the duration of treatment. Treatment continued for about six months, at which point a wash-out period was initiated. Termination of 4-AP treatment led to a rapid relapse of symptom intensity, suggesting that 4-AP treatment was effective in alleviating symptoms.

On 10 mg twice daily of 4AP, the Subject reported significant reduction in all symptoms, allowing resumption of participation in family and avocational activities for the first time in 4 years. Side effects (initial blurring of vision, insomnia, and dyspepsia) were mild. Given these clinical results, a prospective, double blind case study was performed.

Study Design and Procedures

A single subject, prospective, double blind, placebo controlled trial of 4AP was then conducted in this individual. IRB approvals were obtained from all participating institutions and Subject gave written informed consent for all parts of the study. After a 4-week medication washout period, the Subject was randomly assigned a week's course of placebo, followed by a weeklong medication washout period, followed by another week on 4-AP (10 mg twice daily). Based on the medication half-life, a one week washout period was presumed to be sufficient. Subject and assessors were blinded to treatment allocation.

Procedures

Measurements conducted at identical times at baseline and on each of the subsequent 3 weeks of the study included:
Balance and mobility assessed by Hi-Mat test (Arch. Phys. Med. Rehabil. 2006, 87(3), 437-442; hereby incorporated by reference in its entirety).
Subjective symptom assessment using the Rivermead Post-Concussion Questionnaire (Clin. Rehabil. 2005, 19(8), 878-887; hereby incorporated by reference in its entirety).
Neuropsychological assessment, consisting of the digit span and cancellation tests from the Wechsler Adult Intelligence Scale, 4^{th} edition (Coalson, D. L., Engi-Raiford, S. Wechsler Adult Intelligence Scale, 4^{th} Ed., in Technical and Interpretive Manual, 4^{th} Ed., San Antonio: Pearson (2008); hereby incorporated by reference in its entirety), the California Verbal Learning Test II (CVLT) (Delis, D. C., Kramer, J. H., Kaplan, E., Ober, B. A. in CVLT-II Manual, San Antonio: The Psychological Corporation (2000); hereby incorporated by reference in its entirety), the Verbal Fluency Test (Connors Continuous Performance Test Computer II Program Version 5 Users Manual, Toronto: Multi-Health Systems, Inc. (2002); hereby incorporated by reference in its entirety), and the Connors Continuous Performance Test (J. Nervous Ment. Dis. 1985, 173(6), 353-357; hereby incorporated by reference in its entirety).
Videonystagmography (VNG) and rotary chair (RC) vestibular testing

During testing sessions, study personnel noted relapse of paroxysmal grimacing, weakness, sialorrhea, slurred speech, and slowed responses that the Subject had experienced prior to starting 4AP. In an effort to corroborate the presence of abnormal movements, video recordings of identical testing segments were reviewed by raters, physical, occupational, and speech therapists all with experience working with TBI. Raters were blinded to subject history and study variables. Absent any established rating scales for the unusual phenomenon observed, an abbreviated version of the Abnormal Involuntary Movement Scale (AIMS) was used (Prog. Brain Res. 2007, 161, 327-338; hereby incorporated by reference in its entirety).

Based on the observation of significant central findings on videonystagmography and rater-observed reduction in abnormal movements while on the active medication, the Subject was asked to return for 2 additional rounds of open-label testing, 5 and 9 weeks after conclusion of the formal study. The Subject resumed taking 4AP 10 mg twice daily immediately after the conclusion of the blinded testing.

Results

Results of testing are presented in Table 3. Balance/mobility, symptoms and neuropsychological evaluations were similar during all 4 blinded phases of testing. An improvement in AIMS was observed in the active medication phase compared to placebo and washout conditions.

TABLE 3
Results of Testing

| | 1- | 2- | 3- | 4- | 5- | 6- |
|---|---|---|---|---|---|---|
| Test Session | Baseline | Placebo | Washout | Active | 1^{st} Open | 2^{nd} Open |
| HiMat score | 18 | 28 | 28 | 31 | 32 | 50 |
| Rivermead score | 43 | 24 | 44 | 41 | 21 | 7 |
| Cancellation task | 37 | 37 | 37 | 37 | 75 | 84 |
| percentile | | | | | | |
| CVLT-II | 27 | 21 | 65 | 18 | 69 | 93 |
| immediate, | | | | | | |
| percentile | | | | | | |
| AIMS Subscale | na | 15.0 | 9.5 | 4.5 | na | na |
| Left Dix-Hallpike | 4°/sec left | 5°/sec left | 3°/sec left | 4°/sec left | 1°/sec left | 1°/sec left* |

na = data not available for that session;
CVLT = California Verbal Learning Test;
AIMS = Abnormal Involuntary Movement Scale;
*after 50 second delay

On rotary chair testing, phase leads, gains, asymmetry, fixation (at 0.16 Hz, 0.64 Hz), rotational trapezoid testing, and subjective visual vertical components were all normal. On VNG testing, calorics for all 4 conditions was symmetric and within range of normal, as were saccades, smooth pursuit, optokinetic testing, right dix hallpike, and right head positioning. Left dix hallpike positioning provoked immediate left beating, non-fatiguing nystagmus on all four tests with degrees/second of nystagmus measured as 4, 5, 3, and 4 on tests 1-4 each. On head left positioning, left beating, non-fatiguing nystagmus was demonstrated measuring 6, 2, 2 and 4 degrees/second on test 1-4. Down beating nystagmus with head tilted down was present on all tests.

In the two open phases of the study, balance/mobility and subjective symptoms demonstrated improvement during extended medication use. The Cancellation and CVLT also demonstrated improved cognitive processing speed and auditory memory with extended medication use. VNG on test 5 showed 1 degree/second of non-fatiguing left beating nystagmus in left dix hallpike, and 2 degrees/second of left beating non-fatiguing nystagmus in head left position. On test 6, left dix hallpike testing elicited 1 degree/second of left beating nystagmus, but only after patient was in position for 50 seconds.

Non-fatiguing left-beating nystagmus on head left and after left dix-hallpike positioning, and persistent DBN when head tilted down are all indicative of central pathology. The reduction in degree of nystagmus after left dix-hallpike in tests 5 and 6, and the 50 second delay of onset in test 6, suggest an ameliorative medication effect. This is consistent with findings in mobility, neuropsychological, and subjective assessments on tests 5 and 6, as well as the retrospective assessment by blinded raters who noted improvement when subject was on active medication. These findings suggest possible effectiveness of 4AP for PCS, but with a longer onset of action than originally assumed.

Neuro-optometric evaluation revealed reduced eyestrain/headache/and tracking problems when reading; decreased dizziness, fatigue, sleep disturbance, and seizure-like activity; improvements in sensitivity to sound, auditory discrimination, concentration ability, memory, and motor coordination. Overall, neuro-optometric functioning was unexpectedly improved following treatment of 4AP.

Neurofeedback evaluation also revealed dramatic and unexpected improvements in neurofeedback parameters such as, for example, neurosynchronization and restoration of brain activity.

### Example 2

### A Diagnostic Method for Identifying, Classifying or Diagnosing Subject Suffering from PCS Who May be Candidates for Treatment with 4-AP

Relevant patients will have a history of a non-penetrating head injury or whiplash injury, with or without consciousness loss, stemming from a traumatic event that occurred at least 48 hours prior to evaluation to generate symptoms within Clusters A-D, defined in Example 1. Symptoms may be chronic or intermittent, but regardless of their presentation, should contribute to or cause a clear decrement in the patient's premorbid level of functioning and quality of life.

The patient should be given a complete physical exam, including a detailed medical history and review of systems, along with blood tests including a complete blood count, liver function tests (e.g. AST and ALT), electrolytes, and toxicology screen to ensure that no concurrent medical conditions underlie the symptoms associated with PCS. Ideally, the patient should also be examined by a neurologist and have an MRI of the head to likewise rule out concurrent and confounding conditions. The presence of concurrent conditions in no way diminishes the effectiveness of the diagnostic method described herein, and it should be understood that PCS patients having such concurrent conditions will benefit from 4-AP therapy provided the prescribed diagnostic criteria are met.

To assess balance impairments within Cluster A, a standardized assessment of balance should be administered. A preferred standardized balance assessment is the Berg Balance Test (See Berg, K. O. et al. Arch. Phys. Med. Rehabil. 2004, 85, 1128-1135; incorporated herein by reference in its entirety). The Berg Balance test was developed to measure balance among older people with impairment in balance function by assessing the performance of functional tasks. It is a valid instrument used for evaluation of the effectiveness of interventions and for quantitative descriptions of function in clinical practice and research. Patients are asked to complete fourteen tasks while an examiner rates the patient's performance on each task. Elements of the test are representative of daily activities that require balance, such as sitting, standing, leaning over, and stepping. Some tasks are rated according to the quality of the performance of the task, while others are evaluated by the time required to complete the task.

Although results of the Berg Balance Test are usually reported as a score, the actual score is not as important for the present invention as is the evaluation and observation of a patient's performance of the tasks. For example, the Berg test may be used to determine whether a PCS patient possesses difficulties in maintaining stability with eyes closed, conversing while walking, or difficulty in reaching forward or overhead with an outstretched arm while standing—symptoms that, when present, suggest a patient will be responsive to 4-AP treatment. Used in this way, the Berg Balance Test is used to identify areas of impairment and the effects of the impairment on function.

A rigorous neurological examination and verbal questioning should be sufficient to evaluate symptoms in Cluster B associated with vertigo. The presence of vestibular seizures, in particular, is a critical symptom within Cluster B. Generally speaking, persistent sensations, such as being off balance or tilted, or feeling of motion after coming to a rest, are Cluster B symptoms that are likely to be treatable with 4-AP administration. If available, examination by a neuro-otologist will be useful in assessing the presence of balance abnormalities.

Several tests are available to assess symptoms in Cluster C. A preferred assessment is the Rivermead Post-Concussion Symptoms Questionnaire (“RPQ”) (See King, N. S. et al. J. Neurol. 1995, 242, 587-592; incorporated herein by reference in its entirety). The RPQ is administered to someone who sustains a concussion or other form of traumatic brain injury to measure the severity of symptoms, and is widely used to determine the presence and severity of post-concussion syndrome (PCS). The test asks patients to rate the severity of 16 different symptoms commonly found after a mild traumatic brain injury over the past 24 hours on a scale from 0 to 4: absent, mild, moderate, or severe. In each case, the symptom is compared with how severe it was before the injury occurred.

In addition, and if available, the patient should receive a neuropsychological assessment of memory, concentration, speed and efficiency of mental processing, and verbal fluency. One preferred assessment in determining impairments in Cluster C is Connors' Continuous Performance Test II. This is a computer-based program from the Multi-Health Systems Corporation. The test is a measure of sustained attentional capacity, and evaluates several levels of sustained attention, including aspects of inattention and impulsivity. The test-retest correlation coefficients have been shown to be highly satisfactory. It has also been shown to be relatively unaffected by practice effects.

Two sub-tests from the Wechsler Adult Intelligence Scale-Fourth Edition (WAIS-IV, 2008) are also preferred to examine deficits within Cluster C: Cancellation and Digit Span. The Cancellation sub-test evaluates visual processing speed and visual scanning, and is highly related to other tests of processing speed and moderately correlated with measures of working memory. The Digit Span sub-test measures aspects of immediate or simple attention and auditory working memory. Both Wechsler sub-tests can be used to evaluate whether deficits exist in registration or retention of newly presented information and overall speed of mental processing.

It is anticipated that other tests known to those of skill in the art may be useful in determining whether a patient possesses the cognitive or emotional impairments characteristic of Cluster C. For example, the California Verbal Learning Test-II, a test of auditory memory and recall, and the Controlled Oral Word Association test, a measure of verbal fluency and productivity, are disclosed herein as being useful for this purpose.

Determining the presence and severity of most symptoms in Cluster D may be achieved by means of a thorough physical examination of the patient. However, assessment of symptoms associated with visual impairments may require examination by a neurophthalmologist. The particular visual impairments in a patient suffering from PCS that are likely to be addressable by 4-AP treatment are related to functioning of the oculomotor system, including an inability or impairment in tracking objects in motion and nystagmus.

Once it has been established that a PCS patient exhibits serious deficits within two or more, preferably three or more Clusters, that patient is likely to respond positively to 4-AP therapy.

Beneficial results from treatment with 4-AP may be observable as an decrease in the severity or disappearance of symptoms in one or more, preferably two or more, and more preferably three or more Clusters. Such differences may be quantifiable by comparing scores of administered tests obtained during treatment with tests administered prior to treatment. For example, follow-up neuropsychological testing showing consistent improvement by one standard deviation of scores obtained on cognitive testing is considered a significant response. Likewise, documented improvement on ophthalmological or neuro-otological testing of vision or balance is considered a significant response. In the presence of a significant response, and absent any adverse reactions, treatment with 4-AP (or the effective agent utilized) should be continued.

### Example 3

### Diagnostic Method for Identifying, Classifying or Diagnosing Subject Suffering from PCS Who May be Candidates for Treatment with 4-AP

While it is fully understood that observation of symptoms within Clusters A-D defined above is sufficient to identify PCS patients likely to benefit from therapeutic treatment with 4-AP and similar compounds, it may be desirable to more precisely define this class of patient. For instance, the need for more precision may arise in the context of designating inclusion criteria for a clinical trial of an identified medications thought to be effective in treating symptoms of this nature. In addition, a more precisely defined population of PCS patient may be useful in screening compounds or agents effective to relieve symptoms. Finally, this defined population may be useful in identifying genetic mutations that might predispose a head trauma patient to display chronic PCS symptoms.

To achieve this goal, a point system was applied to discrete symptoms within symptom Clusters to specifically define a class of PCS patient that will be likely to benefit from treatment from 4-AP. Data from evaluations documenting symptoms prior to therapy are shown in Table 2. According to the weighted point system, symptoms from each cluster are further assigned a point value. The frequency of each symptom over the span of about one week prior to the assessment was rated according to the following scale: 0—Never present, 1—Sometimes present, 2—Often present, 3—Always or Nearly always present. The rating number for each item was recorded as the raw score, and the raw score was then multiplied by the weight factor to obtain the weighted score. The total weighted score of symptoms exhibited prior to therapy was 165.

TABLE 2
PCS Symptom Cluster Scale - Prior to Therapy

| | | Raw | | Weighted |
|---|---|---|---|---|
| | Symptom | score | Weight | Score |
| Cluster A | Difficulty in standing for more than 5 minutes | 0 | 2 | 0 |
| | Inability to maintain stability while standing with | 2 | 3 | 6 |
| | eyes closed or with feet together | | | |
| | Impairment of gait | 2 | 3 | 6 |
| | Difficulty moving body position (e.g., from chair to | 1 | 1 | 1 |
| | bed or toilet) | | | |
| | Difficulty rising unaided from a sitting position or | 2 | 1 | 2 |
| | from sitting on the floor | | | |
| | Inability to maintain heel-to-toe gait | 3 | 1 | 3 |
| | Difficulty maintaining balance while looking over | 2 | 2 | 4 |
| | shoulder | | | |
| | Difficulty conversing while walking | 3 | 3 | 9 |
| | Difficulty tossing objects from hand to hand while | 3 | 1 | 3 |
| | walking | | | |
| Cluster B | Vertigo | 2 | 2 | 6 |
| | Persistent sensation of being off-balance or tilted | 3 | 4 | 12 |
| | Persistent sensation of walking on soft or moving | 2 | 3 | 6 |
| | surface | | | |
| | Persistence or perseveration of proprioceptive | 2 | 4 | 8 |
| | sensations (e.g., feeling motion after coming to rest) | | | |
| | Oscillopsia (perception of stationary objects | 2 | 4 | 8 |
| | moving) | | | |
| | Vestibular seizures | 2 | 5 | 10 |
| Cluster C | Persisting difficulty with registration or retention of | 2 | 2 | 4 |
| | newly presented information in any modality | | | |
| | Inability to tolerate distraction (e.g., inability to | 3 | 3 | 9 |
| | complete simple task or resume conversation after | | | |
| | interruption) | | | |
| | Inability to perform two simple tasks concurrently | 3 | 3 | 9 |
| | (“multitasking”) | | | |
| | Slowed speed of mental processing or responding | 3 | 2 | 6 |
| | Emotional lability or exaggerated emotions | 1 | 2 | 2 |
| | Persistent or frequent irritability | 1 | 2 | 2 |
| | Apathy, listlessness, blunting of affect | 1 | 2 | 2 |
| | Intolerance of noise, light, or commotion | 3 | 4 | 12 |
| Cluster D | Headache | 1 | 2 | 2 |
| | Insomnia | 2 | 1 | 2 |
| | Anergy, chronic fatigue, hypersomnia | 2 | 3 | 6 |
| | Difficulty in accommodation or focus | 3 | 3 | 9 |
| | Diplopia | 0 | 2 | 0 |
| | Nystagmus | 1 | 4 | 4 |
| | Impairment of tracking an object in motion | 3 | 4 | 12 |
| Total | | | | 165 |

Following therapy, re-evaluation of the subject was performed using the same scale, and the data are presented in Table 3. The total weighted score of symptoms exhibited after therapy was 33.

TABLE 3
PCS Symptom Cluster Scale - After Therapy with 4-AP

| | | Raw | | Weighted |
|---|---|---|---|---|
| | Symptom | score | Weight | Score |
| Cluster A | Difficulty in standing for more than 5 minutes | 0 | 2 | 0 |
| | Inability to maintain stability while standing with | 1 | 3 | 3 |
| | eyes closed or with feet together | | | |
| | Impairment of gait | 0 | 3 | 0 |
| | Difficulty moving body position (e.g., from chair to | 0 | 1 | 0 |
| | bed or toilet) | | | |
| | Difficulty rising unaided from a sitting position or | 0 | 1 | 0 |
| | from sitting on the floor | | | |
| | Inability to maintain heel-to-toe gait | 1 | 1 | 1 |
| | Difficulty maintaining balance while looking over | 0 | 2 | 0 |
| | shoulder | | | |
| | Difficulty conversing while walking | 1 | 3 | 3 |
| | Difficulty tossing objects from hand to hand while | 1 | 1 | 1 |
| | walking | | | |
| Cluster B | Vertigo | 0 | 2 | 0 |
| | Persistent sensation of being off-balance or tilted | 1 | 4 | 4 |
| | Persistent sensation of walking on soft or moving | 0 | 3 | 0 |
| | surface | | | |
| | Persistence or perseveration of proprioceptive | 0 | 4 | 0 |
| | sensations (e.g., feeling motion after coming to rest) | | | |
| | Oscillopsia (perception of stationary objects | 0 | 4 | 0 |
| | moving) | | | |
| | Vestibular seizures | 0 | 5 | 0 |
| Cluster C | Persisting difficulty with registration or retention of | 1 | 2 | 2 |
| | newly presented information in any modality | | | |
| | Inability to tolerate distraction (e.g., inability to | 1 | 3 | 3 |
| | complete simple task or resume conversation after | | | |
| | interruption) | | | |
| | Inability to perform two simple tasks concurrently | 1 | 3 | 3 |
| | (“multitasking”) | | | |
| | Slowed speed of mental processing or responding | 0 | 2 | 0 |
| | Emotional lability or exaggerated emotions | 0 | 2 | 0 |
| | Persistent or frequent irritability | 0 | 2 | 0 |
| | Apathy, listlessness, blunting of affect | 0 | 2 | 0 |
| | Intolerance of noise, light, or commotion | 1 | 4 | 4 |
| Cluster D | Headache | 2 | 2 | 4 |
| | Insomnia | 2 | 1 | 2 |
| | Anergy, chronic fatigue, hypersomnia | 0 | 3 | 0 |
| | Difficulty in accommodation or focus | 1 | 3 | 3 |
| | Diplopia | 0 | 2 | 0 |
| | Nystagmus | 0 | 4 | 0 |
| | Impairment of tracking an object in motion | 0 | 4 | 0 |
| Total | | | | 33 |

In this study, the raw data from evaluations documenting symptoms prior to therapy and after treatment with 4-AP were used. The subject in this study scored a total of 165 points prior to initiating therapy with 4-AP. After a course of treatment with 4-AP, the subject was re-evaluated according to the same point scale and scored a total of 33 points following treatment.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

1. A method for treating or suppressing post concussion syndrome in a subject in need thereof, which comprises administering to the subject a therapeutically effective amount of a compound or a pharmaceutically acceptable salt, solvate, or hydrate thereof, or a composition comprising a compound or a pharmaceutically acceptable salt, solvate, or hydrate thereof and a pharmaceutically acceptable carrier, wherein the compound modulates or blocks neuronal ion channel function.

2. The method of claim 1, wherein the ion channel is a voltage-gated potassium channel.

3. The method of claim 1, wherein the compound is a mono- or di-aminopyridine.

4. The method of claim 1, wherein the compound is 3,4-diaminopyridine or 4-aminopyridine.

5. The method of claim 4, wherein the compound is 4-aminopyridine.

6. The method of claim 1, wherein the subject exhibits symptoms selected from at least two symptom clusters selected from the group consisting of balance impairments, vertigo, cognitive or emotional impairments, and somatosensory impairments.

7. The method of claim 1, wherein the subject exhibits symptoms selected from at least three symptom clusters selected from the group consisting of balance impairments, vertigo, cognitive or emotional impairments, and somatosensory impairments.

8. The method of claim 1, wherein the subject exhibits at least one symptom from each of the following symptom clusters: balance impairments, vertigo, cognitive or emotional impairments, and somatosensory impairments.

9. The method of claim 1, wherein the therapeutically effective amount of the compound is between from about 1 mg/day to about 100 mg/day.

10. The method of claim 1, wherein the therapeutically effective amount of the compound is between from about 5 mg/day to about 30 mg/day.

11. The method of claim 1, wherein the therapeutically effective amount of the compound is between from about 10 mg/day to about 20 mg/day.

12. The method of claim 1, wherein the subject has partially responded to treatment for traumatic brain injury.

13. The method of claim 1, wherein the subject has not responded to treatment for traumatic brain injury.

14. The method of claim 1, wherein the subject is suffering from treatment resistant post concussion syndrome.

15. The method of claim 1, wherein administration is oral.

16. The method of claim 1, wherein the subject exhibits symptoms of post-traumatic stress disorder, vertigo, depression, or substance abuse.

17. A method for treating or suppressing post concussion syndrome in a subject in need thereof, which comprises a) determining that the subject exhibits symptoms selected from at least two system clusters selected from the group consisting of symptoms related to balance impairments, symptoms related to vertigo, symptoms related to cognitive or emotional impairments, and symptoms related to somatosensory impairments; and b) administering to the subject a therapeutically effective amount of a compound or a pharmaceutically acceptable salt, solvate, or hydrate thereof, or a composition comprising a compound or a pharmaceutically acceptable salt, solvate, or hydrate thereof and a pharmaceutically acceptable carrier, wherein the compound modulates or blocks neuronal ion channel function.

18. The method of claim 17, wherein the ion channel is a voltage-gated potassium channel.

19. The method of claim 17, wherein the compound is a mono- or di-aminopyridine.

20. The method of claim 19, wherein the compound is 3,4-diaminopyridine or 4-aminopyridine.

21. The method of claim 20, wherein the compound is 4-aminopyridine.

22. The method of claim 17, wherein the subject exhibits symptoms selected from at least three symptom clusters.

23. The method of claim 17, wherein the subject exhibits at least one symptom from each symptom cluster.

24. The method of claim 17, wherein the therapeutically effective amount of the compound is between from about 1 mg/day to about 100 mg/day.

25. The method of claim 17, wherein the therapeutically effective amount of the compound is between from about 5 mg/day to about 30 mg/day.

26. The method of claim 17, wherein the therapeutically effective amount of the compound is between from about 10 mg/day to about 20 mg/day.

27. The method of claim 17, wherein the subject has partially responded to treatment for traumatic brain injury.

28. The method of claim 17, wherein the subject has not responded to treatment for traumatic brain injury.

29. The method of claim 17, wherein the subject is suffering from treatment resistant post concussion syndrome.

30. The method of claim 17, wherein administration is oral.

31. A method for identifying, classifying or diagnosing post concussion syndrome, comprising determining that the subject exhibits symptoms selected from at least two system clusters selected from the group consisting of symptoms related to balance impairments, symptoms related to vertigo, symptoms related to cognitive or emotional impairments, and symptoms related to somatosensory impairments, wherein the determination is performed by a physical or neurological examination of the subject.

32. The method of claim 31, further comprising the step of administering to the subject a therapeutically effective amount of a compound or a pharmaceutically acceptable salt, solvate, or hydrate thereof, or a composition comprising a compound or a pharmaceutically acceptable salt, solvate, or hydrate thereof and a pharmaceutically acceptable carrier, wherein the compound modulates or blocks neuronal ion channel function.

33. The method of claim 32, wherein said subject exhibits a reduction of said symptoms following administration of said compound or composition.

34. The method of claim 32, wherein the compound is 4-aminopyridine or 3,4-diaminopyridine.

35. The method of claim 34, wherein the compound is 4-aminopyridine.

36. The method of claim 32, wherein the symptoms related to balance impairments are selected from the group consisting of difficulty in standing for more than 5 minutes, inability to maintain stability while standing with eyes closed or with feet together, impairment of gait on a smooth surface, impairment of gait on an uneven surface, difficulty moving body position, difficulty rising unaided from a sitting position or from sitting on a floor, difficulty in reaching forward or overhead with an outstretched arm while standing, inability to maintain heel-to-toe gait, difficulty maintaining balance while looking over one shoulder, difficulty conversing while walking, and difficulty tossing objects from hand to hand while walking.

37. The method of claim 32, wherein the symptoms related to vertigo are selected from the group consisting of vertigo, persistent sensation of being off-balance or tilted, persistent sensation of walking on soft or moving surfaces, persistence or perseveration of proprioceptive sensations, oscillopsia, and vestibular seizures.

38. The method of claim 32, wherein the symptoms related to cognitive or emotional impairments are selected from the group consisting of persistent difficulty with registration or retention of newly presented information in any modality, distractibility, inability to tolerate distraction, impairment of judgment, inability to perform two simple tasks concurrently, slowed speed of mental processing or responding, emotional lability or exaggerated emotions, persistent or frequent irritability, and apathy, listlessness, or blunting of affect.

39. The method of claim 32, wherein the symptoms related to somatosensory impairments are selected from the group consisting of headache, insomnia, anergy, chronic fatigue or hypersomnia, difficulty in accommodation or focus, diplopia, nystagmus, impairment of tracking an object in motion, and intolerance of noise, light or commotion.

40. The method of claim 32, wherein the subject has experienced head or brain injury prior to determination of symptoms.

41. The method of claim 32, wherein the subject has partially responded to previous treatment for traumatic brain injury.

42. The method of claim 32, wherein the subject has not responded to previous treatment for traumatic brain injury.

43. The method of claim 32, wherein symptoms are assigned a point total according to frequency and weighted to obtain a weighted score.

44. The method of claim 32, further comprising the step of evaluating subject's response to administration, wherein said subject exhibits a reduction of symptoms following administration of said compound or composition.

45. The method of claim 44, wherein symptoms are assigned a point total according to frequency and weighted to obtain a weighted score.

46. The method of claim 45, wherein the weighted score after administration is reduced by greater than about 20% relative to the weighted score prior to administration.

47. The method of claim 45, wherein the weighted score after administration is reduced by greater about 50% relative to the weighted score prior to administration.

48. The method of claim 45, wherein the weighted score after administration is reduced by greater about 60% relative to the weighted score prior to administration.

49. The method of claim 45, wherein the weighted score after administration is reduced by greater about 70% relative to the weighted score prior to administration.

50. The method of claim 45, wherein the weighted score after administration is reduced by greater about 80% relative to the weighted score prior to administration.

51. A kit for evaluating a subject for post concussion syndrome comprising,
a) a list of symptom clusters,
b) a compound or composition comprising a compound or a pharmaceutically acceptable salt, solvate or hydrate thereof and a pharmaceutically acceptable carrier, wherein the compound modulates or blocks neuronal ion channel function, and
(c) instructions for identifying, classifying, diagnosing or evaluating the subject.

52. The kit of claim 51, wherein the compound is a mono- or di-aminopyridine.

53. The kit of claim 52, wherein the compound is 3.4-diaminopyridine or 4-aminopyridine.

54. The kit of claim 53, wherein the compound is 4-aminopyridine.
